(19) United States
(12) Patent Application Publication
(10) Pub. No.: US 2025/0116554 A1
(43) Pub. Date: Apr. 10, 2025

(54) SYSTEMS AND METHODS FOR TOOTH SHADE IMAGING IN DENTAL TREATMENT

(71) Applicant: ALIGN TECHNOLOGY, INC., San Jose, CA (US)

(72) Inventors: Ofer SAPHIER, Rehovot (IL); Shai FARKASH, Hod Hasharon (IL)

(73) Assignee: ALIGN TECHNOLOGY, INC., San Jose, CA (US)

(21) Appl. No.: 18/901,423

(22) Filed: Sep. 30, 2024

Related U.S. Application Data
(60) Provisional application No. 63/621,037, filed on Jan. 15, 2024.
(60) Provisional application No. 63/542,829, filed on Oct. 6, 2023.

(51) Int. Cl. G01J 3/50; A61B 1/24; A61C 13/34

(52) U.S. Cl. CPC G01J 3/508; A61B 1/24; A61C 13/34

(57) ABSTRACT

A system for tooth shade imaging in dental treatment may include one or more processors and memory having instructions that when executed by the one or more processors may cause the system to receive scan data of a patient's dentition, determine that a tooth of interest for shade capture is within a field of view of a shade capture device, capture a first shade of the tooth of interest before tooth preparation, capture a second shade of the tooth of interest after tooth preparation, generate a digital model of the tooth of interest after tooth preparation, and model a third shade resulting from placing a prosthetic on the tooth of interest after tooth preparation using the digital model of the tooth of interest after tooth preparation including the second shade and a digital model of the prosthetic.

### BACKGROUND

This application claims the benefit under 35 U.S.C. § 119 (e) of U.S. Provisional Patent Application No. 63/542,829, filed Oct. 6, 2023, and titled “SYSTEMS AND METHODS FOR TOOTH SHADE IMAGING IN DENTAL TREATMENT,” and U.S. Provisional Patent Application No. 63/621,037, filed Jan. 15, 2024, and titled “SYSTEMS AND METHODS FOR TOOTH SHADE IMAGING IN DENTAL TREATMENT,” which is incorporated, in its entirety, by this reference.

Determining the correct shade for dental restorations, such as crowns, bridges, or veneers, is a challenging task for a number of reasons. Natural teeth aren't monochromatic and can have multiple shades, translucencies, and intrinsic characteristics that can be difficult to capture with current imaging devices and techniques and to replicate with dental materials.

The light under which the tooth shade is selected can significantly influence the appearance of the tooth. Different lights (LED, fluorescent, incandescent, natural daylight) can make the same tooth appear as a different shade. Lack of control over the lighting during tooth shade determination also leads to incorrect or unnaturally looking dental restorations. Similarity, two objects can appear to match under one light source or lighting condition but look different under another light source or in another lighting condition, known as metamerism. Matching shades of teeth can be problematic if a shade is chosen in one lighting condition, but the restoration is viewed in another.

One method of determining a tooth shade is through the use of shade guides. However, even if a matching shade guide appears close in shade to a tooth, slight variations can exist between the shade tab and the patient's natural tooth. Moreover, shade guides also may not match the translucency, absorption, scattering, and other material properties of a patient's natural tooth, leading to incorrect shading of a restoration.

Even after a shade is chosen, different dental materials used to fabricate the restoration, such as ceramics or composites, have different optical properties from each other and from a patient's teeth. A shade that works well with one material might not look the same or work as well with another.

Current shade imaging and determination systems are less than idea for at least the above described reasons. Work in relation to the present disclosure has found that current intraoral scanners have been produce inaccurate and shading results. For example, they fail to separate reflected light, which is mainly the color of the light source, from diffused light, which is mainly the color of the object. They also do not account for lateral and axial nonuniformity of the tooth shade when light and/or the imaging device is off-axis, too far, or too close to the teeth. As another example, single purpose shade devices also lack the ability to account for the uncertainties of lighting, tooth material properties, and the other factors discussed above. They also are not connected to the dental treatment systems to properly and accurately identify the teeth, leading to less than ideal shades in dental restorations and user error.

### SUMMARY

As will be described in greater detail below, the present disclosure describes various systems and methods for imaging and determining the shades of a patient's teeth.

### INCORPORATION BY REFERENCE

All patents, applications, and publications referred to and identified herein are hereby incorporated by reference in their entirety, and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a block diagram of an example system for determining the shade of a patient's teeth, in accordance with some embodiments.

FIG. 2 shows aspects of the system of FIG. 1 in use in scanning and determining the shade of a patient's teeth, in accordance with some embodiments.

FIG. 3 shows a method of determining the shade of a patient's teeth, in accordance with some embodiments.

FIG. 4 shows an intraoral scanning probe sleeve for use in determining the shade of a patient's teeth, in accordance with some embodiments.

FIG. 5 shows an image of a field of view of an intraoral scanning probe for determining the shade of a patient's teeth, in accordance with some embodiments.

FIG. 6 shows a block diagram of an example system for photo guidance, in accordance with some embodiments.

FIG. 7 shows a flow diagram of example method for photo guidance, in accordance with some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

### Virtual Care System

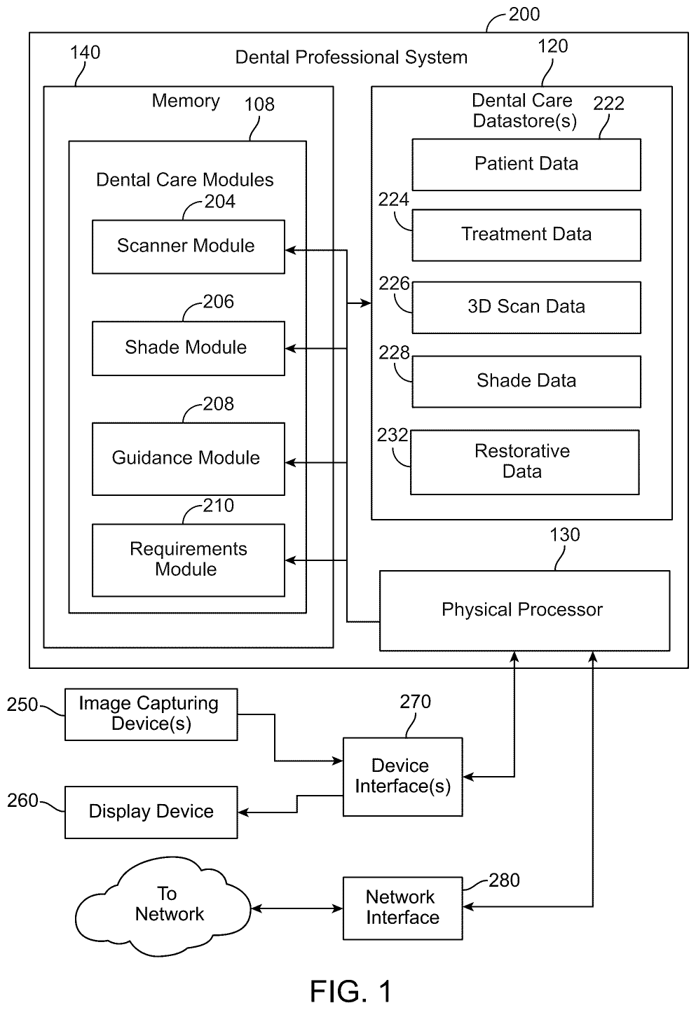

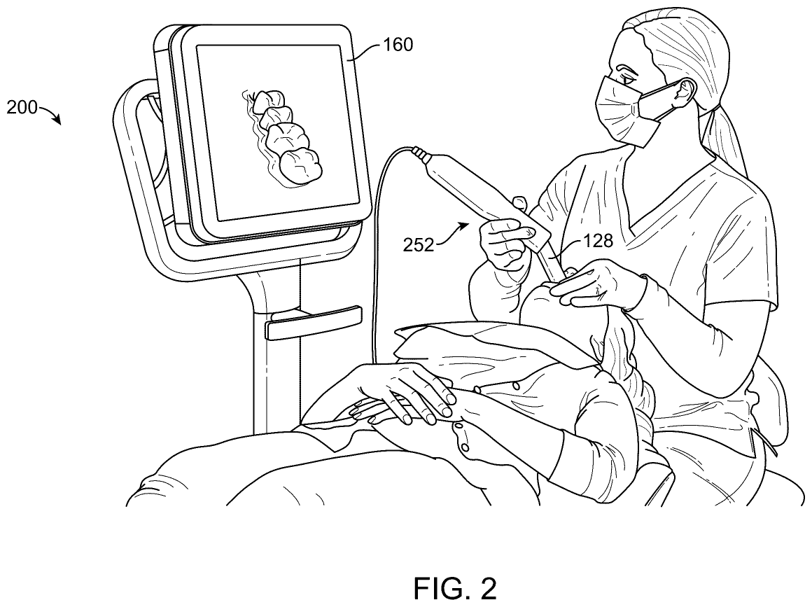

With reference to FIGS. 1 and 2, an example dental professional system 200 for determining the shade of teeth is shown, in accordance with some embodiments. FIG. 1, depicts a block diagram of the system 200. The dental professional system 200 generally represents any type or form of computing device capable of reading computer-executable instructions. Dental professional system 200 may be, for example, a desktop computer, a tablet computing device, a laptop, a smartphone, an augmented reality device, or other consumer device. Additional examples of dental professional system 200 include, without limitation, dental scanning systems, laptops, tablets, desktops, servers, cellular phones, Personal Digital Assistants (PDAs), multimedia players, embedded systems, wearable devices (e.g., smart watches, smart glasses, etc.), smart vehicles, smart packaging (e.g., active or intelligent packaging), gaming consoles, Internet-of-Things devices (e.g., smart appliances, etc.), variations or combinations of one or more of the same, and/or any other suitable computing device.

In various implementations, the dental professional system 200 is configured to interface with a dental professional. A “dental professional” (used interchangeably with dentist, orthodontist, and doctor herein) as used herein, may include any person with specialized training in the field of dentistry, and may include, without limitation, general practice dentists, orthodontists, dental technicians, dental hygienists, etc. A dental professional may include a person who can assess, diagnose, and/or treat a dental condition. “Assessment” of a dental condition, as used herein, may include an estimation of the existence of a dental condition. An assessment of a dental condition need not be a clinical diagnosis of the dental condition. In some embodiments, an “assessment” of a dental condition may include an image-based shade assessment, that is an assessment of a shade of a patient's tooth or dentition based in part or on whole on photos and/or images (e.g., images that are not used to stitch a mesh or form the basis of a clinical scan) taken of the dental condition. A “diagnosis” of a dental condition, as used herein, may include a clinical identification of the nature of an illness or other problem by examination of the symptoms. “Treatment” of a dental condition, as used herein, may include prescription and/or administration of care to address the dental conditions. Examples of treatments to dental conditions include prescription and/or administration of brackets/wires, clear aligners, and/or other appliances to address orthodontic conditions, prescription and/or administration of restorative elements, such as crowns, bridges, and veneers, to address functional and/or aesthetic aspects of the patient's dentition, etc. The dental professional system 200 may provide to a user software (e.g., one or more webpages, standalone applications (e.g., dedicated treatment planning and/or treatment visualization applications), mobile applications, etc.) that allows the user to interact with patient data, treatment data, scan data, shade data, prosthetic data, treatment planning tools, control image capturing devices, the display of information, etc.

As illustrated in FIG. 1, the dental professional system 200 may include one or more memory devices, such as memory 140. Memory 140 generally represents any type or form of volatile or non-volatile storage device or medium capable of storing data and/or computer-readable instructions. In one example, memory 140 may store, load, execute in conjunction with physical processor(s) 130, and/or maintain one or more of dental care modules 108. Examples of memory 140 include, without limitation, Random Access Memory (RAM), Read Only Memory (ROM), flash memory, Hard Disk Drives (HDDs), Solid-State Drives (SSDs), optical disk drives, caches, variations, or combinations of one or more of the same, and/or any other suitable storage memory.

As illustrated in FIG. 1, dental professional system 200 may also include one or more physical processors, such as physical processor(s) 130. Physical processor(s) 130 generally represents any type or form of hardware-implemented processing unit capable of interpreting and/or executing computer-readable instructions. In one example, physical processor(s) 130 may access and/or modify one or more of dental care modules 108 stored in memory 140. Additionally or alternatively, physical processor 130 may execute one or more of dental care modules 108 to facilitate determining the shade of a patient's teeth. Examples of physical processor(s) 130 include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, and/or any other suitable physical processor.

In some embodiments, the dental professional system 200 may include one or more image capturing devices 250. Image capturing devices 250 may comprise a camera, scanner, or other optical sensor. Image capturing devices 250 may include one or more lenses, one or more camera devices, and/or one or more other optical sensors. In some examples, image capturing devices 250 may include other sensors and/or devices which may aid in capturing optical data, such as one or more lights, depth sensors, etc. In some examples, the image capturing devices 205 may include a near-infrared imaging device that is sensitive to near-infrared wavelengths of light. In some embodiments, the image capturing devices 250 may include a filter, such as a near-infrared band-pass filter that allows passage of near-infrared light and blocks light in the visible and/or far infrared wavelengths.

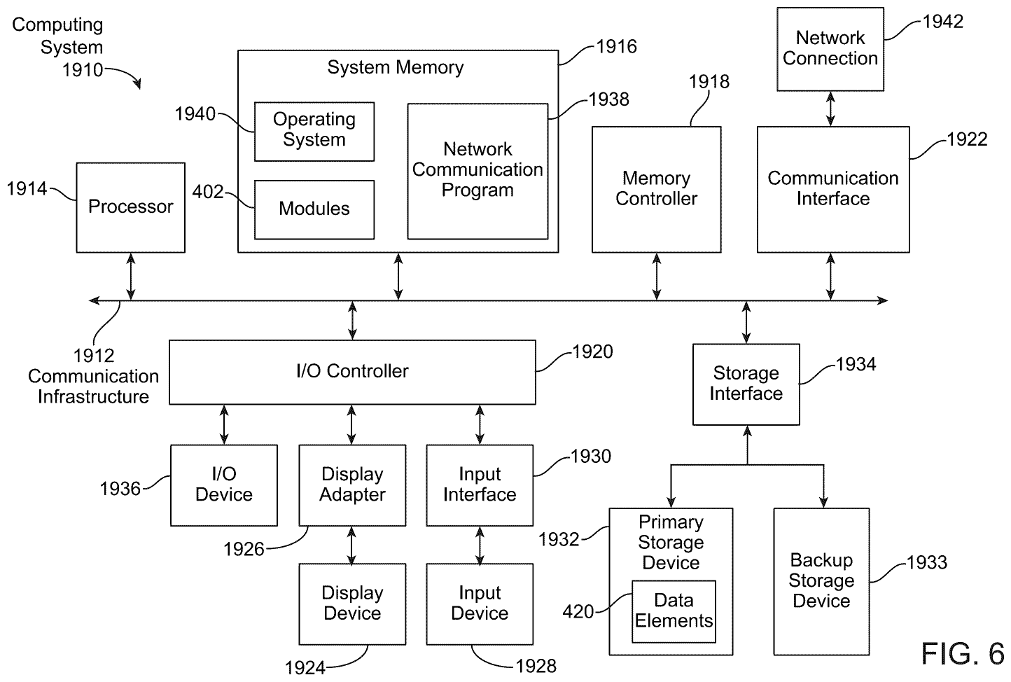

Network interface 280 may include an interface for communication between the system 200 and its physical processor 130 and a network. The network may a computer network that facilitates communication or data transfer using wireless and/or wired connections. Examples of a network include, without limitation, an intranet, a Wide Area Network (WAN), a Local Area Network (LAN), a Personal Area Network (PAN), the Internet, Power Line Communications (PLC), a cellular network (e.g., a Global System for Mobile Communications (GSM) network), portions of one or more of the same, variations or combinations of one or more of the same, and/or any other suitable network. The network may also comprise a connection between elements inside a single device (e.g., a bus, any communications infrastructure (e.g., communications infrastructure 1912 shown in FIG. 6, etc.).

Dental care datastore(s) 120 includes one or more datastore configured to store any type or form of data that may be used for dental care. In some embodiments, the dental care datastore(s) 120 include, without limitation, patient data 222 and treatment data 224. Patient data 222 may include data collected from patients, such as patient dentition information, patient historical data, patient scans, patient information, etc. Patient data may include information related to which teeth the patient may be present or missing. Patient data 222 may include data to identity the patient, such as their name, age, and other information.

Treatment data 224 may include the tooth or teeth selected for restoration, the type of restoration, such as a crown, bridge, or veneer, and other information related to the treatment of the patient, such as their dental treatment prescription. In some embodiments, treatment data 224 may include data used for treating patients, such as treatment plans, state of treatment, success of treatment, changes to treatment, notes regarding treatment, etc. The treatment plan data 224 may include the positions and orientations of each of the patient's teeth for each stage of a treatment plan. In some embodiments, the positions and orientations of the teeth may be stored as three-dimensional positional locations and angular orientations of each tooth and the patient's upper and lower arches. In some embodiments the positions and orientations of the patient's teeth may be stored as a collection of three-dimensional segmented models of the patient's upper and lower arches for each stage of treatment.

Patient data and treatment data may also include one or more of 3D scan data 226, shade data 228, restorative data 232.

The dental care datastore(s) 120 may include 3D scan data 226 which may include data representing three-dimensional models the patient's dentition, including the teeth and gingiva. In some embodiments, the 3D scan data 226 may include segmented 3D models of the patient's detention wherein each individual tooth of the patient and/or the gingiva is separated from the other teeth and/or gingiva of the detention. The three-dimensional models may be generated based on an initial three-dimensional (or 3D) intraoral scan of the patient's teeth. As shown in FIG. 2, during an intraoral scan, an image capture device, such as a handheld scanning device 252 generates a three-dimensional model of the patient's upper and lower arches. After capturing three-dimensional models of the upper and lower arches, each tooth within the three-dimensional model is separated from the model to form an individual tooth model. These individual tooth models are then used during the orthodontic treatment planning process in order to generate each of the of treatment stages to move the teeth from an initial position towards a target final position and then to generate orthodontic aligners that are worn on the patient's teeth in order to move the teeth from the initial position towards the final position. The 3D scan data may also be used during restorative treatment and treatment planning to determine the size and shape of the external surfaces of the restorative prosthetic, such as the crown, bridge, or veneer. The external surfaces may include the occlusal, lingual, buccal, and interproximal surface of the crown, as well as the intaglio surface shape that matches the shape of a prepared tooth in the 3D scan data.

The dental care datastore(s) 120 may include shade data 228. Shade data may be two-dimensional (or 2D) color images which may include data representing two-dimensional images of the patient's mouth and teeth. In some embodiments, the two-dimensional images are captured using the systems and methods described herein, for example by using an image capture device 250, discussed herein. The two-dimensional images may include buccal images of the patient's teeth.

The dental care datastore(s) 120 may include restorative data 232 may include data relating to the physical properties, appearance, and shape of a restorative. For example, restorative data 232 may include the size and shape of the external surfaces of the restorative prosthetic, such as the crown, bridge, or veneer. The external surfaces may include the occlusal, lingual, buccal, and interproximal surface of the crown, as well as the intaglio surface shape that matches the shape of a prepared tooth. Restorative data may also include the shade, transparency, thickness, materials, and other properties of the restoration to be used to treat the patient.

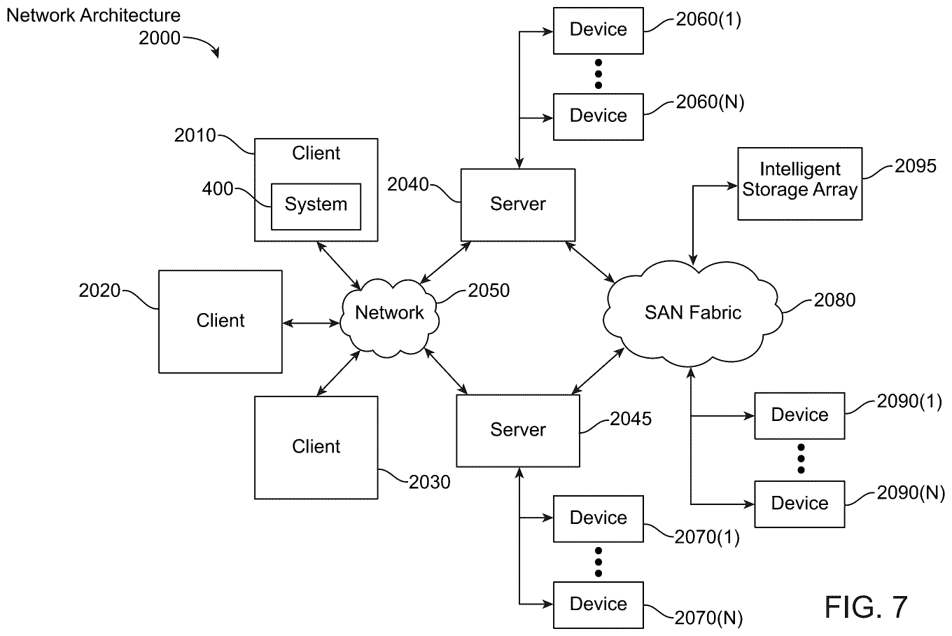

Example system 200 in FIG. 1 may be implemented in a variety of ways. For example, all or a portion of example system 200 may represent portions of example system 200 in FIG. 2, system 1900 in FIG. 6, or system 2000 in FIG. 7.

As will be described in greater detail below, one or more of dental care modules 108 and/or the dental care datastore(s) 120 in FIG. 1 may, (when executed by at least one processor of dental professional system 200) enable dental professional system 200 to facilitate providing shade capture and analysis. Shade capture and analysis as used herein, may include computer-program instructions and/or software operative to provide shade capture and analysis by a health professional (dentist, orthodontist, dental technician, etc.) to a patient, a potential consumer of dental services, and/or other individual. As an example, shade capture and analysis may include software operative to providing shade capture and analysis by the dental professional system 200 over the network.

In some embodiments, the elements of the system 200 (e.g., the dental care modules 108 and/or the dental care datastore(s) 120) may be operative to provide shade capture and analysis to aid in the capture of images relevant to shade capture and analysis using the image capture device 250 on the dental professional system 200.

The system 200 may include a network interface 280 and a device interface 270. The network interface 280, which may be coupled to a network and to the processor 130, may transmit signals to and receive signals from other wired or wireless devices, including remote (e.g., cloud-based) storage devices, cameras, and/or displays. For example, the network interface 280 may include wired (e.g., serial, ethernet, or the like) and/or wireless (Bluetooth, Wi-Fi, cellular, or the like) transceivers that may communicate with any other feasible device through any feasible network.

The device interface 270, which is coupled to the processor 130, may be used to interface with any feasible input and/or output device. For example, the device interface 250 may be coupled to and interface with one or more image capturing devices 130. Example image capturing devices may include 3D scanners, such as intraoral 3D scanners, optical cameras, x-ray devices, panoramic x-ray devices, shade capture devices, or the like. In another example, the device interface 270 may be coupled to and interface with a display device 260. Through the display device 260, the processor 260 may display images, feedback information, instructions, or the like.

In some examples, the display device 260 may be an integral part of the device 200. In other words, the display device 260 may share a common housing or enclosure with the processors, data stores, etc.

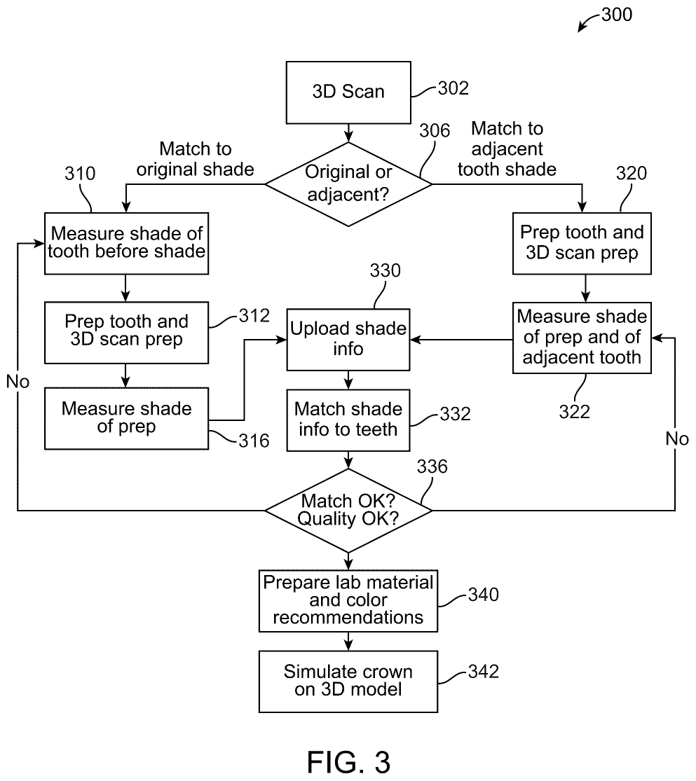

With reference to FIG. 3, a method 300 for shade capture and analysis is shown, in accordance with some embodiments. The method 300 may begin at block 302 with a 3D scan of the patient's dentition. At block 302, a 3D model of the patient's detention may be received or generated. The dental professional may use an image capture device 250, such as a 3D intraoral scanner 252 to generate a 3D model of the patient's detention. The 3D model may be generated by capturing a series of images of the patient's detention, generating a 3D point cloud for each of the series of images, registering the 3D point clouds together to form a 3D model of the patient's detention, and then segmenting the dental objects within the 3D model. Segmenting the dental objects may include separating each of the patient's teeth into an individual tooth model with a position and location that corresponds to its position and orientation in the 3D model. Segmenting may also include segmenting prepared teeth. In some embodiments, segmenting may also include segmenting the gingiva and/or other soft tissue surfaces.

The image capture device 250 may also capture near-infrared data, which penetrates into the tooth. Near-infrared data may include information related to the transparency and internal structure of the tooth. The near-infrared data may be aligned and/or mapped onto the 3D model.

The image capture device 250 may also capture color data of the surface of the patient's dentition. The surface color may be texture mapped to the 3D model.

At block 306 the system may receive an input for the type of shade capture and analysis to be performed. In some embodiments, the input may be a procedure type, such as crown, bridge, or veneer. In some embodiments, the input may include a status of the tooth for which the shade capture and analysis is to be performed. For example, if the tooth has already been prepared, then the shade may be matched to an adjacent tooth. In some embodiments, a tooth to be restored may have an undesirable shade, for example, the tooth may be dead, in which case capturing and analysis may result in shading a restored tooth to the shade of a dead tooth. In some embodiments, the input may be in the form of a prescription or treatment plan that includes information as to the type of tooth shade to be captured and analyzed, such as an original shade or an adjacent tooth shade. If the capture and analysis is to the original shade, then the process proceeds to block 310. If the capture and analysis is to the shade of one or more adjacent teeth, then the process proceeds to block 320.

At block 310 the original shade of the tooth is captured. The shade may be measured as discussed herein with a second image capture device 250, such as a shade capture device may be a 2D image capture device. A shade taking device, is preferably calibrated to work at a specific range of distances from the patient's teeth and a specific range of angles to the tooth, such as the surface normal of the tooth. The 2D image capture device may not have 3D measurement capabilities. Accordingly, is may not be able to estimate its distance and angle with respect to the tooth based on just the 2D images or without additional information, such as the 3D model of the patient's dentition, as discussed herein.

As discussed herein, with knowledge of the 3D shape of the tooth, such as the 3D model of the patient's detention, the position, distance, angle and orientation of the shade capture device may be determined based on the 2D images of the shade-taking device, where we are relative to the tooth. If the distance and angle are too far from accepted positions, the system can provide feedback to the user about it, such as to ask them to retake the image of the tooth or teeth or how to adjust the position of the shade capture device to capture the tooth or teeth.

In some embodiments, if the difference from desired distance and angle is small, the differences between the measured shade and the actual shade may be compensated for. For example, if the distance is off by 5% or less, the illumination brightness will different than the calibrated brightness and the captured shade may be compensated for based on the difference in distance for example, by increasing or decreasing the brightness.

The image capture device 250 may be guided to the correct tooth for shade capture and analysis. The image capture device 250 may begin capturing images of the patient's dentition. The teeth in the images may be compared to the teeth in the treatment plan, such as in a 3D model of the teeth captured at block 302.

In some embodiments, the comparison of the teeth in the image, which may be a 2D image, may be aligned with a projection of the teeth in the 3D model. For example, the system may receive camera data, such as the focal length, focal distance, field of view, image sensor size, etc. The process may generate multiple 2D projections of the 3D model from different angles, distances, etc, based on the camera data. Outlines of the teeth in the 2D projection of the 3D model may be generated and compared to generated outlines of the teeth in the 2D image. When the system finds a match between the teeth in the 3D model (or the 2D projection thereof) and the teeth in the 2D image, the system may determine that the tooth for which shade capture and analysis is desired, is within the field of view of the shade imaging device and begin capturing shade data. If the field of view of the imaging device does not contain the tooth of interest, then feedback may be provided to the user to guide the movement of the imaging device and its field of view towards the tooth of interest. Feedback may include guidance, such as arrow or other directions on the display, indicating a direction to move the image capture device.

In some embodiments, the teeth in the field of view of the imaging device may also be determined based on matching a shape of the tooth silhouette, the shape the gingiva, such as the gingival margin, the incisal edges of the teeth, and/or the color or color patterns of the teeth in the 2D image with the teeth in the 3D model, with or without projecting the 3D model into a 2D image plane. In some embodiments, the system may receive input from a user of the tooth for which shade capture and analysis is to be performed. For example, the images may be captured by the image capture device and a user may touch the display or click with an input device on the tooth. In some embodiments, an indication of an area, such as by drawing a circle or enclosed shaped on the image or images of the teeth.

At block 312 the tooth selected for restoration is prepared. Tooth preparation may include a material removal operation to shape the tooth to receive the restoration. After preparation, the tooth may be scanned with a 3D scanner to determine the final shape of the prepared tooth. The final shape of the prepared restoration may be used in preparing the size and shape of the physical restoration.

At block 316 the shade of the prepared tooth may be captured. During treatment, the restoration is placed over the prepared tooth. The transparency of the material used to make the restoration may allow a portion of the shade of the prepared tooth to impact the apparent shade of the restored tooth. For example, some of the color of the prepared tooth may be visible though the preparation.

The shade of the prepared tooth may be measured as discussed herein with an image capture device 250. The image capture device 250 may be guided to the correct prepared tooth for shade capture and analysis. The image capture device 250 may begin capturing images of the patient's dentition. The teeth in the images may be compared to the teeth in the treatment plan, such as in a 3D model of the teeth captured at block 302.

In some embodiments, the comparison of the teeth in the image, which may be a 2D image, may be aligned with a projection of the teeth in the 3D model. For example, the system may receive camera data, such as the focal length, focal distance, field of view, image sensor size, etc. The process may generate multiple 2D projections of the 3D model from different angles, distances, etc, based on the camera data. Outlines of the teeth in the 2D projection of the 3D model may be generated and compared to generated outlines of the teeth in the 2D image. When the system finds a match between the teeth in the 3D model (or the 2D projection thereof) and the teeth in the 2D image, the system may determine that the tooth for which shade capture and analysis is desired, is within the field of view of the shade imaging device and begin capturing shade data. If the field of view of the imaging device does not contain the prepared tooth, then feedback may be provided to the user to guide the movement of the imaging device and its field of view towards the prepared tooth. Feedback may include guidance, such as arrow or other directions on the display, indicating a direction to move the image capture device to capture the prepared tooth.

In some embodiments, the teeth in the field of view of the imaging device may also be determined based on matching a shape of the prepared tooth silhouette, the shape the gingiva, such as the gingival margin, the incisal edges of the teeth, and/or the color or color patterns of the teeth in the 2D image with the teeth in the 3D model, with or without projecting the 3D model into a 2D image plane. In some embodiments, the system may receive input from a user of the tooth for which shade capture and analysis is to be performed. For example, the images may be captured by the image capture device and a user may touch the display or click with an input device on the prepared tooth. In some embodiments, an indication of an area, such as by drawing a circle or enclosed shaped on the image or images of the teeth.

At block 330 the shade information may be updated based on the shade of the tooth before preparation, the shade of the prepared tooth, the position and shape of the prepared tooth, the position and shape of the teeth adjacent the prepared tooth, transparency of the tooth, the surface texture of the original tooth surface before the preparation, and other factors.

At block 320 the tooth selected for restoration is prepared. Tooth preparation may include a material removal operation to shape the tooth to receive the restoration. After preparation, the tooth may be scanned with a 3D scanner to determine the final shape of the prepared tooth. The final shape of the prepared restoration may be used in preparing the size and shape of the physical restoration.

At block 322 the shade of the prepared tooth and teeth adjacent the prepared tooth may be captured. The shade of the teeth may be measured as discussed herein with an image capture device 250. The image capture device 250 may be guided to the correct tooth for shade capture and analysis. The image capture device 250 may begin capturing images of the patient's dentition. The teeth in the images may be compared to the teeth in the treatment plan, such as in a 3D model of the teeth captured at block 302.

In some embodiments, the comparison of the teeth in the image, which may be a 2D image, may be aligned with a projection of the teeth in the 3D model. For example, the system may receive camera data, such as the focal length, focal distance, field of view, image sensor size, etc. The process may generate multiple 2D projections of the 3D model from different angles, distances, etc, based on the camera data. Outlines of the teeth in the 2D projection of the 3D model may be generated and compared to generated outlines of the teeth in the 2D image. When the system finds a match between the teeth in the 3D model (or the 2D projection thereof) and the teeth in the 2D image, the system may determine that the teeth for which shade capture and analysis is desired are within the field of view of the shade imaging device and begin capturing shade data of the teeth. If the field of view of the imaging device does not contain the teeth of interest, then feedback may be provided to the user to guide the movement of the imaging device and its field of view towards the teeth of interest. Feedback may include guidance, such as arrow or other directions on the display, indicating a direction to move the image capture device to capture the teeth of interest.

In some embodiments, the teeth in the field of view of the imaging device may also be determined based on matching a shape of the tooth silhouette, the shape the gingiva, such as the gingival margin, the incisal edges of the teeth, and/or the color or color patterns of the teeth in the 2D image with the teeth in the 3D model, with or without projecting the 3D model into a 2D image plane. In some embodiments, the system may receive input from a user of the tooth for which shade capture and analysis is to be performed. For example, the images may be captured by the image capture device and a user may touch the display or click with an input device on the teeth. In some embodiments, an indication of an area, such as by drawing a circle or enclosed shaped on the image or images of the teeth.

At block 330 the shade information may be updated based on the shade of the tooth before preparation, the shade of the prepared tooth, the position and shape of the prepared tooth, the position and shape of the teeth adjacent the prepared tooth, transparency of the tooth, the surface texture of the original tooth surface before the preparation, and other factors.

At block 332 the shade information is matched to the teeth of the patient. For example, the shade information may be associated with a tooth in the treatment plan or the prescription. The shade may also be matched or compared with the teeth in the 3D scan of the patient's dentition.

At block 336 the shade match and quality of the shade images are evaluated. For example, the focus, brightness, angle of the camera with respect to the tooth surface normal, distance from the tooth being too far, etc may be evaluated based on the 2D images and the 3D model, such as by deriving the location and orientation of the shade imaging device with respect to the tooth of interest based on a comparison of the teeth in the shade image with the teeth in the 3D model. If the shade and quality are acceptable, then the process may proceed to block 340. If the shade and/or quality are not acceptable, then the process may go back to blocks 310 or 320. In some embodiments, while the process may have proceeded through blocks 310, 312, and 316 in a first pass, the process may proceed to blocks 320 and 322 in a second pass. For example, because the original surface shape of the tooth may no longer exists, after preparation, the adjacent teeth may be used to determine the shade of the restoration.

At block 340 a digital model of the prepared tooth and a prosthetic is generated. For example, the digital model of the prepared tooth may be based on the scan of the prepared tooth, such as generated at block 312 or 320. The 3D model of the prosthetic may be based on the thickness of the restoration and the design of the external surface of the crown and the 3D scan of the prepared tooth. In some embodiments, material properties, such as transparency, light scattering, etc for a plurality of wavelengths of light may be applied to the volume of the restoration. In some embodiments, a material and the prosthetic fabrication may be selected and the prosthetic may be modeled based on the material and in some embodiments, also based on the fabrication method. For example, the material may be a single shade monoblock, a multi shade monoblock, such as a three-shed monoblock or a sintering material. A monoblock may be milled. Sintering material may be sintered.

The transparency of the material may be modeled based on the choice of material and the material's transparency, thickness, the shade of the preparation, and the desired restoration shade based on the shade determined according to this process, such as the shade of the tooth the restoration is restoring or the teeth adjacent to the tooth the restoration is restoring.

The shade may also be modeled based on the surface texture of the original tooth or the adjacent teeth. The texture, such as the fine 3D surface roughness, and features may be determined based on the 3D scan of the patient's original tooth or the adjacent teeth.

At block 342 the shade of the restoration is simulated based on the model generated at block 340. The simulation may be a ray tracing or other light and optical simulation of light illuminating the restoration and the rest of the patient's dentition.

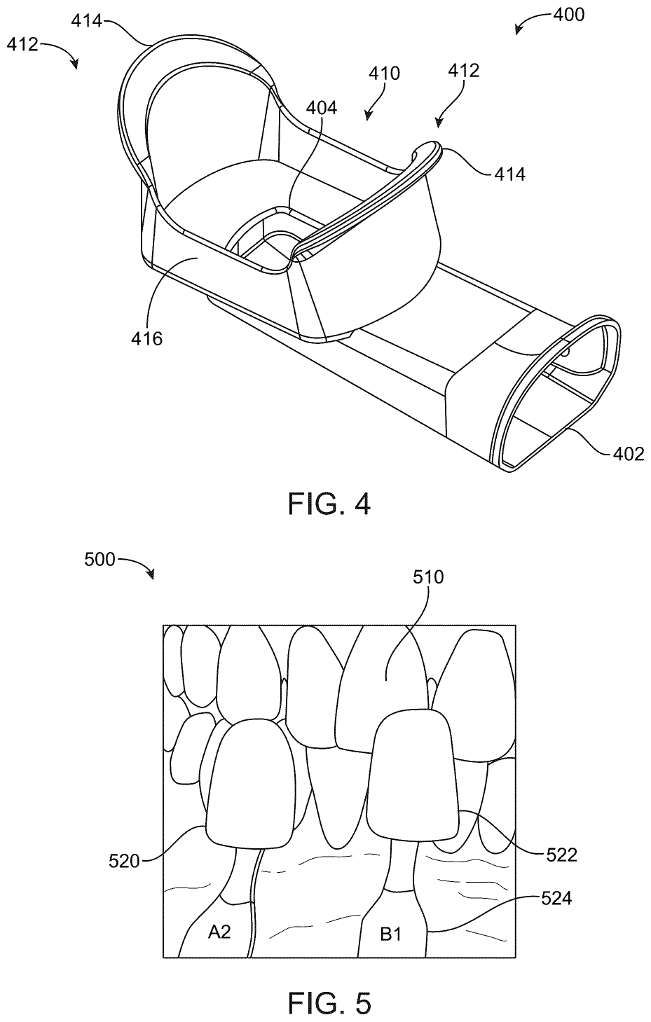

With reference to FIG. 4, a sleeve for adapting a 3D scanner for capturing shade is shown, in accordance with some embodiments, herein. The sleeve 400 may have an aperture or a channel 402 to receive the distal end 128 of an intraoral scanning probe 252 (see FIG. 2) which includes the imaging portion of the intraoral scanning portion. The imaging portion of an intraoral scanning probe, by which light is emitted and the reflected light is received may be directed out of the aperture 404 with the distal end of the intraoral scanning probe is inserted into the sleeve 400. The sleeve 400 includes a lip retracting shade 410 that extends from a distal end of the sleeve 400 and around the aperture 404. The lip retracting shade 410 serves multiple purposes. The proximal and distal wings 412 include sidewalls that flair outward, away from the aperture 404, to capture and aid in retracting the lips of the patient to keep them away from the teeth that are being scanned and, when the lips are closed around the lip retracting shade 410, ambient light is blocked from entering the oral cavity during shade image capture. The lip retracting shade 410 may include one or more cutouts 416 in the perimeter of the shade. The cutouts may allow for insertion of shade references, such as shade tabs, to be inserted into the field of view of the scanner when the scanner is capturing shade images, for example, as shown in FIG. 5.

FIG. 5 depicts a field of view of an intraoral scanner during shade image capture. The image 500 includes the target tooth 510. The treatment plan may include a restoration for the target tooth 510 for which a shade is desired. The image 500 also includes two shade references 520 and 522 of known shades. By capturing the shade reference 520 and 522 under the same controlled lighting conditions provided by LEDs or other calibrated lights sources of the intraoral scanner and the shade provided by the sleeve 400 as the target tooth 510, the shade of the target tooth can be determined.

In some emblements, the intraoral scanner may be used as the shade capture device used in process 300. When using an intraoral scanner, the 3D scanning functions of the scanner may be used along with 2D shade image capture. The dental professional system 200 may switch between 3D scanning and shade capture functions automatically, based on a process flow of the system or may receive an input to change from 3D scanning to 2D shade image capture.

The shade tabs and their labels may be automatically identified by the dental professional system in the images captured by the intraoral scanner through image segmentation, wherein the edges of the gingiva, teeth, and shade tabs and determined and then the images segmented. The shades of the shade references, such as the shade tabs may be determined based on character recognition of the shade ID 524 on the shade tabs.

In some embodiments, a 3D scan of the teeth may be conducted close in time with the shade image capture, such as within 100 ms, either before or after, the shade image capture in order to determine the distance, angle, position, and orientation of the intraoral scanner with respect to the target tooth or teeth.

The system may check that the shade images taken are in a desired distance and angle range with respect to the tooth or teeth.

In some embodiments, images without illumination provided by the intraoral scanner may also be used to estimate ambient background illumination and shade. If strong non uniformity of ambient background illumination is detected, such, for example, when an overhead dental projector is used, but is partly obstructed by a doctor's hand, feedback, such as a warning may be provided on the display.

The system may also display the captured shade images and identity the teeth for which sufficient shade data has been captured and for which insufficient shade data has been captured.

In some embodiments, the system may determine a difference between an estimated shade of the tooth and the shade of the shade references. If the different between the estimated shade of the tooth and the shade of the shade references, the system may provide feedback to the user regarding the difference. In some embodiments, the system may suggest a different shade reference to be used.

The shade images captured by the 3D scanner using the sleeve 400, along with the 3D scan of the patient's dentition, color texture for the 3D scan, near IR data, such as near IR data that includes the location of the dentin in the tooth, transparency, and internal tooth texture and structure, the prescription, and other patient and treatment data, may be provided to a restoration design and fabrication system for use in selecting materials and shades for the restoration, as discussed herein. In some embodiments, low quality images, such as images with poor focus, movement blur, that are too dark or too light, may be removed from the data sent to the restoration design and fabrication system, which may be a remote dental laboratory.

The data may be displayed within dental restoration preparation environment, such as within dental restoration CAD design software. The patient and treatment data may be presented and the tooth being restored may be selected from the 3D model of the patient's donation or from the prescription.

### Computing System

FIG. 6 is a block diagram of an example computing system 1910 capable of implementing one or more of the embodiments described and/or illustrated herein. For example, all or a portion of computing system 1910 may perform and/or be a means for performing, either alone or in combination with other elements, one or more of the steps described herein (such as one or more of the steps illustrated in FIG. 3). All or a portion of computing system 1910 may also perform and/or be a means for performing any other steps, methods, or processes described and/or illustrated herein.

Computing system 1910 broadly represents any single or multi-processor computing device or system capable of executing computer-readable instructions. Examples of computing system 1910 include, without limitation, workstations, laptops, client-side terminals, servers, distributed computing systems, handheld devices, or any other computing system or device. In its most basic configuration, computing system 1910 may include at least one processor 1914 and a system memory 1916.

Processor 1914 generally represents any type or form of physical processing unit (e.g., a hardware-implemented central processing unit) capable of processing data or interpreting and executing instructions. In certain embodiments, processor 1914 may receive instructions from a software application or module. These instructions may cause processor 1914 to perform the functions of one or more of the example embodiments described and/or illustrated herein.

System memory 1916 generally represents any type or form of volatile or non-volatile storage device or medium capable of storing data and/or other computer-readable instructions. Examples of system memory 1916 include, without limitation, Random Access Memory (RAM), Read Only Memory (ROM), flash memory, or any other suitable memory device. Although not required, in certain embodiments computing system 1910 may include both a volatile memory unit (such as, for example, system memory 1916) and a non-volatile storage device (such as, for example, primary storage device 1932, as described in detail below). In one example, one or more of dental care modules 108 from FIG. 1 may be loaded into system memory 1916.

In some examples, system memory 1916 may store and/or load an operating system 1940 for execution by processor 1914. In one example, operating system 1940 may include and/or represent software that manages computer hardware and software resources and/or provides common services to computer programs and/or applications on computing system 1910. Examples of operating system 1940 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 1910 may also include one or more components or elements in addition to processor 1914 and system memory 1916. For example, as illustrated in FIG. 6, computing system 1910 may include a memory controller 1918, an Input/Output (I/O) controller 1920, and a communication interface 1922, each of which may be interconnected via a communication infrastructure 1912. Communication infrastructure 1912 generally represents any type or form of infrastructure capable of facilitating communication between one or more components of a computing device. Examples of communication infrastructure 1912 include, without limitation, a communication bus (such as an Industry Standard Architecture (ISA), Peripheral Component Interconnect (PCI), PCI Express (PCIe), or similar bus) and a network.

Memory controller 1918 generally represents any type or form of device capable of handling memory or data or controlling communication between one or more components of computing system 1910. For example, in certain embodiments memory controller 1918 may control communication between processor 1914, system memory 1916, and I/O controller 1920 via communication infrastructure 1912.

I/O controller 1920 generally represents any type or form of module capable of coordinating and/or controlling the input and output functions of a computing device. For example, in certain embodiments I/O controller 1920 may control or facilitate transfer of data between one or more elements of computing system 1910, such as processor 1914, system memory 1916, communication interface 1922, display adapter 1926, input interface 1930, and storage interface 1934.

As illustrated in FIG. 6, computing system 1910 may also include at least one display device 1924 coupled to I/O controller 1920 via a display adapter 1926. Display device 1924 generally represents any type or form of device capable of visually displaying information forwarded by display adapter 1926. Similarly, display adapter 1926 generally represents any type or form of device configured to forward graphics, text, and other data from communication infrastructure 1912 (or from a frame buffer, as known in the art) for display on display device 1924.

As illustrated in FIG. 6, example computing system 1910 may also include at least one input device 1928 coupled to I/O controller 1920 via an input interface 1930. Input device 1928 generally represents any type or form of input device capable of providing input, either computer or human generated, to example computing system 1910. Examples of input device 1928 include, without limitation, a keyboard, a pointing device, a speech recognition device, variations, or combinations of one or more of the same, and/or any other input device.

Additionally or alternatively, example computing system 1910 may include additional I/O devices. For example, example computing system 1910 may include I/O device 1936. In this example, I/O device 1936 may include and/or represent a user interface that facilitates human interaction with computing system 1910. Examples of I/O device 1936 include, without limitation, a computer mouse, a keyboard, a monitor, a printer, a modem, a camera, a scanner, a microphone, a touchscreen device, variations, or combinations of one or more of the same, and/or any other I/O device.

Communication interface 1922 broadly represents any type or form of communication device or adapter capable of facilitating communication between example computing system 1910 and one or more additional devices. For example, in certain embodiments communication interface 1922 may facilitate communication between computing system 1910 and a private or public network including additional computing systems. Examples of communication interface 1922 include, without limitation, a wired network interface (such as a network interface card), a wireless network interface (such as a wireless network interface card), a modem, and any other suitable interface. In at least one embodiment, communication interface 1922 may provide a direct connection to a remote server via a direct link to a network, such as the Internet. Communication interface 1922 may also indirectly provide such a connection through, for example, a local area network (such as an Ethernet network), a personal area network, a telephone or cable network, a cellular telephone connection, a satellite data connection, or any other suitable connection.

In certain embodiments, communication interface 1922 may also represent a host adapter configured to facilitate communication between computing system 1910 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fiber Channel interface adapters, Ethernet adapters, or the like. Communication interface 1922 may also allow computing system 1910 to engage in distributed or remote computing. For example, communication interface 1922 may receive instructions from a remote device or send instructions to a remote device for execution.

In some examples, system memory 1916 may store and/or load a network communication program 1938 for execution by processor 1914. In one example, network communication program 1938 may include and/or represent software that enables computing system 1910 to establish a network connection 1942 with another computing system (not illustrated in FIG. 6) and/or communicate with the other computing system by way of communication interface 1922. In this example, network communication program 1938 may direct the flow of outgoing traffic that is sent to the other computing system via network connection 1942. Additionally or alternatively, network communication program 1938 may direct the processing of incoming traffic that is received from the other computing system via network connection 1942 in connection with processor 1914.

Although not illustrated in this way in FIG. 6, network communication program 1938 may alternatively be stored and/or loaded in communication interface 1922. For example, network communication program 1938 may include and/or represent at least a portion of software and/or firmware that is executed by a processor and/or Application Specific Integrated Circuit (ASIC) incorporated in communication interface 1922.

As illustrated in FIG. 6, example computing system 1910 may also include a primary storage device 1932 and a backup storage device 1933 coupled to communication infrastructure 1912 via a storage interface 1934. Storage devices 1932 and 1933 generally represent any type or form of storage device or medium capable of storing data and/or other computer-readable instructions. For example, storage devices 1932 and 1933 may be a magnetic disk drive (e.g., a so-called hard drive), a solid-state drive, a floppy disk drive, a magnetic tape drive, an optical disk drive, a flash drive, or the like. Storage interface 1934 generally represents any type or form of interface or device for transferring data between storage devices 1932 and 1933 and other components of computing system 1910. In one example, dental care datastore(s) 120 from FIG. 1 may be stored and/or loaded in primary storage device 1932.

In certain embodiments, storage devices 1932 and 1933 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 1932 and 1933 may also include other similar structures or devices for allowing computer software, data, or other computer-readable instructions to be loaded into computing system 1910. For example, storage devices 1932 and 1933 may be configured to read and write software, data, or other computer-readable information. Storage devices 1932 and 1933 may also be a part of computing system 1910 or may be a separate device accessed through other interface systems.

Many other devices or subsystems may be connected to computing system 1910. Conversely, all of the components and devices illustrated in FIG. 6 need not be present to practice the embodiments described and/or illustrated herein. The devices and subsystems referenced above may also be interconnected in different ways from that shown in FIG. 6. Computing system 1910 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 1910. All or a portion of the computer program stored on the computer-readable medium may then be stored in system memory 1916 and/or various portions of storage devices 1932 and 1933. When executed by processor 1914, a computer program loaded into computing system 1910 may cause processor 1914 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 1910 may be configured as an Application Specific Integrated Circuit (ASIC) adapted to implement one or more of the example embodiments disclosed herein.

FIG. 7 is a block diagram of an example network architecture 2000 in which client systems 2010, 2020, and 2030 and servers 2040 and 2045 may be coupled to a network 2050. As detailed above, all or a portion of network architecture 2000 may perform and/or be a means for performing, either alone or in combination with other elements, one or more of the steps disclosed herein (such as one or more of the steps illustrated in FIG. 3). All or a portion of network architecture 2000 may also be used to perform and/or be a means for performing other steps and features set forth in the instant disclosure.

Client systems 2010, 2020, and 2030 generally represent any type or form of computing device or system, such as example computing system 1910 in FIG. 6. Similarly, servers 2040 and 2045 generally represent computing devices or systems, such as application servers or database servers, configured to provide various database services and/or run certain software applications. Network 2050 generally represents any telecommunication or computer network including, for example, an intranet, a WAN, a LAN, a PAN, or the Internet. In one example, client systems 2010, 2020, and/or 2030 and/or servers 2040 and/or 2045 may include all or a portion of system 200 from FIG. 1 or the other systems and devices disclosed herein.

As illustrated in FIG. 7, one or more storage devices 2060 (1)-(N) may be directly attached to server 2040. Similarly, one or more storage devices 2070 (1)-(N) may be directly attached to server 2045. Storage devices 2060 (1)-(N) and storage devices 2070 (1)-(N) generally represent any type or form of storage device or medium capable of storing data and/or other computer-readable instructions. In certain embodiments, storage devices 2060 (1)-(N) and storage devices 2070 (1)-(N) may represent Network-Attached Storage (NAS) devices configured to communicate with servers 2040 and 2045 using various protocols, such as Network File System (NFS), Server Message Block (SMB), or Common Internet File System (CIFS).

Servers 2040 and 2045 may also be connected to a Storage Area Network (SAN) fabric 2080. SAN fabric 2080 generally represents any type or form of computer network or architecture capable of facilitating communication between a plurality of storage devices. SAN fabric 2080 may facilitate communication between servers 2040 and 2045 and a plurality of storage devices 2090 (1)-(N) and/or an intelligent storage array 2095. SAN fabric 2080 may also facilitate, via network 2050 and servers 2040 and 2045, communication between client systems 2010, 2020, and 2030 and storage devices 2090 (1)-(N) and/or intelligent storage array 2095 in such a manner that devices 2090 (1)-(N) and array 2095 appear as locally attached devices to client systems 2010, 2020, and 2030. As with storage devices 2060 (1)-(N) and storage devices 2070 (1)-(N), storage devices 2090 (1)-(N) and intelligent storage array 2095 generally represent any type or form of storage device or medium capable of storing data and/or other computer-readable instructions.

In certain embodiments, and with reference to example computing system 1910 of FIG. 6, a communication interface, such as communication interface 1922 in FIG. 6, may be used to provide connectivity between each client system 2010, 2020, and 2030 and network 2050. Client systems 2010, 2020, and 2030 may be able to access information on server 2040 or 2045 using, for example, a web browser or other client software. Such software may allow client systems 2010, 2020, and 2030 to access data hosted by server 2040, server 2045, storage devices 2060 (1)-(N), storage devices 2070 (1)-(N), storage devices 2090 (1)-(N), or intelligent storage array 2095. Although FIG. 7 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 2040, server 2045, storage devices 2060 (1)-(N), storage devices 2070 (1)-(N), storage devices 2090 (1)-(N), intelligent storage array 2095, or any combination thereof. All or a portion of one or more of the example embodiments disclosed herein may also be encoded as a computer program, stored in server 2040, run by server 2045, and distributed to client systems 2010, 2020, and 2030 over network 2050.

As detailed above, computing system 1910 and/or one or more components of network architecture 2000 may perform and/or be a means for performing, either alone or in combination with other elements, one or more steps of any of the methods disclosed herein.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of example system 200 in FIG. 1 may represent portions of a cloud-computing or network-based environment. Cloud-computing environments may provide various services and applications via the Internet. These cloud-based services (e.g., software as a service, platform as a service, infrastructure as a service, etc.) may be accessible through a web browser or other remote interface. Various functions described herein may be provided through a remote desktop environment or any other cloud-based computing environment.

In various embodiments, all or a portion of example system 200 in FIG. 1 may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various embodiments, all or a portion of example system 200 in FIG. 1 may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of example system 200 in FIG. 1 may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems, gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

In addition, all or a portion of example system 200 in FIG. 1 may represent portions of, interact with, consume data produced by, and/or produce data consumed by one or more systems for information management. As used herein, the term “information management” may refer to the protection, organization, and/or storage of data. Examples of systems for information management may include, without limitation, storage systems, backup systems, archival systems, replication systems, high availability systems, data search systems, virtualization systems, and the like.

In some embodiments, all or a portion of example system 200 in FIG. 1 may represent portions of, produce data protected by, and/or communicate with one or more systems for information security. As used herein, the term “information security” may refer to the control of access to protected data. Examples of systems for information security may include, without limitation, systems providing managed security services, data loss prevention systems, identity authentication systems, access control systems, encryption systems, policy compliance systems, intrusion detection and prevention systems, electronic discovery systems, and the like.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations, or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third,” etc. may be used herein to describe various layers, elements, components, regions, or sections without referring to any particular order or sequence of events. These terms are merely used to distinguish one layer, element, component, region or section from another layer, element, component, region, or section. A first layer, element, component, region, or section as described herein could be referred to as a second layer, element, component, region, or section without departing from the teachings of the present disclosure.

As used herein, the term “or” is used inclusively to refer items in the alternative and in combination.

As used herein, characters such as numerals refer to like elements.

The present disclosure includes the following numbered clauses.

Clause 1. A system for tooth shade imaging in dental treatment, the system comprising: one or more processors and memory comprising instructions that when executed by the one or more processors cause the system to: receive scan data of a patient's dentition; determine that a tooth of interest for shade capture is within a field of view of a shade capture device; capture a first shade of the tooth of interest before tooth preparation; capture a second shade of the tooth of interest after tooth preparation; generate a digital model of the tooth of interest after tooth preparation; model a third shade resulting from placing a prosthetic on the tooth of interest after tooth preparation using the digital model of the tooth of interest after tooth preparation including the second shade and a digital model of the prosthetic.

Clause 2. The system of clause 1, wherein the instructions that when executed further cause the system to determine a shade of the prosthetic so that the third shade matches the first shade.

Clause 3. The system of clause 1, wherein the instructions to model the third shade include instructions to model the volume of the restoration.

Clause 4. The system of clause 1, wherein the instructions to model the third shade include instructions to model light characteristics of a material of the volume of the restoration.

Clause 5. The system of clause 1, wherein the light characteristics include the transparency of the material of the volume of the restoration.

Clause 6. The system of clause 1, wherein the light characteristics include the light scattering of the material of the volume of the restoration.

Clause 7. The system of any one of clauses 1-6, wherein the instruction to model the third shade include instruction to model the third shade when a plurality of wavelengths of light illuminate the prosthetic on the tooth of interest after tooth preparation.

Clause 8. The system of clause 1, wherein the instructions that determine that a tooth of interest for shade capture is within a field of view of a shade capture device includes instructions to: receive data from the shade capture device; compare the data from the shade capture device to the scan data of the patient's dentition; and identify the tooth of interest for shade capture is within the field of view of the shade capture device based on the comparison.

Clause 9. The system of clause 1, wherein the instructions that determine that a tooth of interest for shade capture is within a field of view of a shade capture device includes instructions to: receive data from the shade capture device; compare the data from the shade capture device to the scan data of the patient's dentition; and identify the teeth within the field of view of the shade capture device based on the comparison; determine whether or not the tooth of interest is identified within the field of view; provide feedback to a user based on the determination.

Clause 10. The system of clause 1, wherein the instructions that when executed by the one or more processors further cause the system to: determine an angle between the shade capture device and the tooth of interest; provide feedback to a user of the shade capture device based on the angle.

Clause 11. The system of clause 1, wherein the feedback includes an indication of how to adjust the position or orientation of the shade capture device is within an angle range of the normal of a surface of the tooth of interest for shade capture.

Clause 12. The system of clause 1, wherein the instructions that when executed by the one or more processors further cause the system to: determine a distance between the shade capture device and the tooth of interest; provide feedback to a user of the shade capture device based on the distance.

Clause 13. The system of clause 12, wherein the feedback includes an indication of how to adjust the position or orientation of the shade capture device is within a distance range of a surface of the tooth of interest for shade capture.

Clause 14. A system for tooth shade imaging in dental treatment, the system comprising: one or more processors and memory that when executed by the one or more processors cause the system to: receive scan data of a patient's dentition; determine a tooth for restoration and a tooth of interest for shade capture; determine that the tooth of interest for shade capture is within a field of view of a shade capture device; capture a first shade of the tooth of interest; capture a second shade of the tooth for restoration after tooth preparation; generate a digital model of the tooth for restoration after tooth preparation; model a third shade resulting from the application of a prosthetic on the tooth for restoration after tooth preparation using the digital model of the tooth for restoration after tooth preparation having the second shade and a digital model of the prosthetic.

Clause 15. The system of clause 14, wherein the instructions that when executed further cause the system to determine a shade of the prosthetic so that the third shade matches the first shade.

Clause 16. The system of clause 14, wherein the instructions to model the shade include instructions to model the volume of the restoration.

Clause 17. The system of clause 14, wherein the instructions to model the shade include instructions to model light characteristics of a material of the volume of the restoration.

Clause 18. The system of clause 14, wherein the light characteristics include the transparency of the material of the volume of the restoration.

Clause 19. The system of clause 114 wherein the light characteristics include the light scattering of the material of the volume of the restoration.

Clause 20. The system of any one of clauses 14-19, wherein the instruction to model the shade include instruction to model the shade when a plurality of wavelengths of light illuminate the prosthetic on the tooth for restoration after tooth preparation.

Clause 21. The system of clause 14, wherein the instructions that determine that a tooth of interest for shade capture is within a field of view of a shade capture device includes instructions to: receive data from the shade capture device; compare the data from the shade capture device to the scan data of the patient's dentition; and identify the tooth of interest for shade capture is within the field of view of the shade capture device based on the comparison.

Clause 22. The system of clause 14, wherein the instructions that determine that a tooth of interest for shade capture is within a field of view of a shade capture device includes instructions to: receive data from the shade capture device; compare the data from the shade capture device to the scan data of the patient's dentition; and identify the teeth within the field of view of the shade capture device based on the comparison; determine whether or not the tooth of interest is identified within the field of view; provide feedback to a user based on the determination.

Clause 23. The system of clause 14, wherein the instructions that when executed by the one or more processors further cause the system to: determine an angle between the shade capture device and the tooth of interest; provide feedback to a user of the shade capture device based on the angle.

Clause 24. The system of clause 23, wherein the feedback includes an indication of how to adjust the position or orientation of the shade capture device is within an angle range of the normal of a surface of the tooth of interest for shade capture.

Clause 25. The system of clause 14, wherein the instructions that when executed by the one or more processors further cause the system to: determine a distance between the shade capture device and the tooth of interest; provide feedback to a user of the shade capture device based on the distance.

Clause 26. The system of clause 25, wherein the feedback includes an indication of how to adjust the position or orientation of the shade capture device is within a distance range of a surface of the tooth of interest for shade capture.

Clause 27. A system for tooth shade imaging in dental treatment, the system comprising: one or more processors and memory that when executed by the one or more processors cause the system to: receive scan data of a patient's dentition; determine that the tooth of interest for shade capture is within a field of view of a shade capture device; automatically determine a location of one or more shade reference aids within a field of view of a shade capture device; capture a first shade of the tooth of interest along with one or more second shades of the one or more shade reference aids; and adjust the first shade based on the one or more second shades.

Clause 28. The system of clause 27, wherein the instructions that automatically determine a location of one or more shade reference aids within a field of view of a shade capture device includes instructions to: identify characters within the field of view of the shade capture device; and determine the location of one or more shade reference aids within a field of view of a shade capture device based on a location of the characters within the field of view.

Clause 29. The system of clause 27, wherein the instructions that automatically determine a location of one or more shade reference aids within a field of view of a shade capture device includes instructions to: segment an area of the shade reference aids within a field of view of a shade capture device.

Clause 30. The system of clause 27, wherein the instructions that when executed by the one or more processors further cause the system to: determine the tooth of interest within a field of view of a shade capture device.

Clause 31. The system of clause 30, wherein the instructions that when executed by the one or more processors further cause the system to: determine a first distance between the shade capture device and the shade reference aids; determine a second distance between the shade capture device and the tooth of interest; and compare a difference between first distance with the second distance to a range; and if the different is outside the range, provide feedback.

Clause 32. The system of clause 30, wherein the instructions that when executed by the one or more processors further cause the system to: determine a first angle between the shade capture device and the shade reference aids; determine a second angle between the shade capture device and the tooth of interest; and compare a difference between first angle with the second angle to a range; and if the different is outside the range, provide feedback.

Clause 33. The system of clause 27, wherein the instructions to capture a first shade of the tooth of interest along with one or more second shades of the one or more shade reference aids includes instructions that when executed by the one or more processors further cause the system to: illuminate the tooth of interest and the one or more shade reference aids with illumination from the shade capture device.

Clause 34. The system of clause 33, wherein the instructions that when executed by the one or more processors further cause the system to: capture a third shade of the tooth of interest along with one or more fourth shades of the one or more shade reference aids without illumination from the shade capture device.

Embodiments of the present disclosure have been shown and described as set forth herein and are provided by way of example only. One of ordinary skill in the art will recognize numerous adaptations, changes, variations, and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A system for tooth shade imaging in dental treatment, the system comprising:
one or more processors and memory comprising instructions that when executed by the one or more processors cause the system to:
receive scan data of a patient's dentition;
determine that a tooth of interest for shade capture is within a field of view of a shade capture device;
capture a first shade of the tooth of interest before tooth preparation;
capture a second shade of the tooth of interest after tooth preparation;
generate a digital model of the tooth of interest after tooth preparation;
model a third shade resulting from placing a prosthetic on the tooth of interest after tooth preparation using the digital model of the tooth of interest after tooth preparation including the second shade and a digital model of the prosthetic.

2. The system of claim 1, wherein the instructions that when executed further cause the system to determine a shade of the prosthetic so that the third shade matches the first shade.

3. The system of claim 1, wherein the instructions to model the third shade include instructions to model the volume of the restoration and to model light characteristics of a material of the volume of the restoration.

4. The system of claim 3, wherein the light characteristics include the transparency and the light scattering the of the material of the volume of the restoration.

5. The system of claim 1, wherein the instructions to model the third shade include instructions to model the third shade when a plurality of wavelengths of light illuminate the prosthetic on the tooth of interest after tooth preparation.

6. The system of claim 1, wherein the instructions that determine that a tooth of interest for shade capture is within a field of view of a shade capture device includes instructions to:
receive data from the shade capture device;
compare the data from the shade capture device to the scan data of the patient's dentition; and
identify the tooth of interest for shade capture is within the field of view of the shade capture device based on the comparison.

7. The system of claim 1, wherein the instructions that determine that a tooth of interest for shade capture is within a field of view of a shade capture device includes instructions to:
receive data from the shade capture device;
compare the data from the shade capture device to the scan data of the patient's dentition; and
identify the teeth within the field of view of the shade capture device based on the comparison;
determine whether or not the tooth of interest is identified within the field of view;
provide feedback to a user based on the determination.

8. The system of claim 1, wherein the instructions that when executed by the one or more processors further cause the system to:
determine an angle between the shade capture device and the tooth of interest;
provide feedback to a user of the shade capture device based on the angle.

9. The system of claim 1, wherein
wherein the feedback includes an indication of how to adjust the position or orientation of the shade capture device is within an angle range of the normal of a surface of the tooth of interest for shade capture.

10. A system for tooth shade imaging in dental treatment, the system comprising:
one or more processors and memory that when executed by the one or more processors cause the system to:
receive scan data of a patient's dentition;
determine a tooth for restoration and a tooth of interest for shade capture;
determine that the tooth of interest for shade capture is within a field of view of a shade capture device;
capture a first shade of the tooth of interest;
capture a second shade of the tooth for restoration after tooth preparation;
generate a digital model of the tooth for restoration after tooth preparation;
model a third shade resulting from the application of a prosthetic on the tooth for restoration after tooth preparation using the digital model of the tooth for restoration after tooth preparation having the second shade and a digital model of the prosthetic.

11. The system of claim 10, wherein the instructions that when executed further cause the system to determine a shade of the prosthetic so that the third shade matches the first shade.

12. The system of claim 10, wherein the instructions to model the shade include instructions to model the volume of the restoration.

13. The system of claim 11, wherein the instructions to model the shade include instructions to model light characteristics of a material of the volume of the restoration.

14. The system of any one of claim 10, wherein the instruction to model the shade include instruction to model the shade when a plurality of wavelengths of light illuminate the prosthetic on the tooth for restoration after tooth preparation.

15. The system of claim 10, wherein the instructions that determine that a tooth of interest for shade capture is within a field of view of a shade capture device includes instructions to:
receive data from the shade capture device;
compare the data from the shade capture device to the scan data of the patient's dentition; and
identify the tooth of interest for shade capture is within the field of view of the shade capture device based on the comparison.

16. A system for tooth shade imaging in dental treatment, the system comprising:
one or more processors and memory that when executed by the one or more processors cause the system to:
receive scan data of a patient's dentition;
determine that the tooth of interest for shade capture is within a field of view of a shade capture device;
automatically determine a location of one or more shade reference aids within a field of view of a shade capture device;
capture a first shade of the tooth of interest along with one or more second shades of the one or more shade reference aids; and
adjust the first shade based on the one or more second shades.

17. The system of claim 16, wherein the instructions that automatically determine a location of one or more shade reference aids within a field of view of a shade capture device includes instructions to:
identify characters within the field of view of the shade capture device; and
determine the location of one or more shade reference aids within a field of view of a shade capture device based on a location of the characters within the field of view.

18. The system of claim 16, wherein the instructions that automatically determine a location of one or more shade reference aids within a field of view of a shade capture device includes instructions to:
segment an area of the shade reference aids within a field of view of a shade capture device.

19. The system of claim 16, wherein the instructions that when executed by the one or more processors further cause the system to:
determine the tooth of interest within a field of view of a shade capture device.

20. The system of claim 19, wherein the instructions that when executed by the one or more processors further cause the system to:
determine a first distance between the shade capture device and the shade reference aids;
determine a second distance between the shade capture device and the tooth of interest; and
compare a difference between first distance with the second distance to a range; and
if the different is outside the range, provide feedback.
